(19) United States
(12) Patent Application Publication
(10) Pub. No.: US 2007/0010460 A1
(43) Pub. Date: Jan. 11, 2007

(54) Multiple sclerosis therapy and diagnosis

(75) Inventors: Rakesh Chibber, London (GB); Russell Hagan, London (GB)

Correspondence Address: NIXON & VANDERHYE, PC, 901 NORTH GLEBE ROAD, 11TH FLOOR, ARLINGTON, VA, 22203, US

(73) Assignee: BTG INTERNATIONAL LIMITED, London (GB)

(21) Appl. No.: 11/472,554

(22) Filed: Jun. 22, 2006

(30) Foreign Application Priority Data
Jun. 22, 2005 (GB) 0512726.1

(51) Int. Cl. A61K 31/704

(52) U.S. Cl. 514/26

(57) ABSTRACT

A method of treatment of Multiple Sclerosis in a subject is disclosed comprising administering to the subject a compound capable of reducing the activity of Core 2 GlcNAc-T. Also disclosed is a method of diagnosing Multiple Sclerosis in a subject and a method of determining the utility of a test substance for use in the treatment of Multiple Sclerosis comprising determining the ability of the substance to inhibit the activity of Core 2 GlcNAc-T.

The present invention relates to treatments for and diagnosis of neuroinflammatory diseases and in particular multiple sclerosis (MS).

Multiple sclerosis is a disease with a significant inflammatory component. The enzyme Core 2 GlcNAc-T is involved in the synthesis of branched chain, O-linked oligosaccharides. Core 2 GlcNAc-T-EC 2.4.1.102 is also known as UDP-GlcNAc:Galβ1,3GalNAc-R (GlcNAc to GalNAc) β-1,6-N-acetylglucosaminyl transferase or Core 2 β-1,6 N-acetylaminotransferase.

Although this enzyme has been implicated in inflammation (WO 0031109), Orlacchio et al (1997) J Neurol Sci. 22; 151(2):177-83 discloses that the level of activity of Core 2 GlcNAc-T is reduced in lymphomonocytes from patients with both relapsing remitting and progressive MS.

The present inventors have now surprisingly determined that Core 2 GlcNAc-T activity is in fact significantly raised in leukocyte preparations containing peripheral blood mononuclear cells (PBMNC) and polymorphonuclear (PMN) leukocytes from sample patients with MS. As Core 2 based oligosaccharides are found inter alia as a component of the ligands of proteins that are thought to mediate aspects of cell adhesion during the inflammatory response, this has implications for increased leukocyte infiltration of tissues in MS. As raised Core 2 GlcNAc-T contributes to increased adhesiveness of leukocytes, the present inventors have determined that lowering the activity of Core 2 GlcNAc-T should tend to normalise adhesiveness of leukocytes, reduce leukocyte extravasation and reduce neuro-inflammation and associated plaques in MS patients.

Accordingly in a first aspect of the invention is provided a method of treatment of Multiple Sclerosis in a subject comprising administering to a subject in need thereof, a therapeutically effective amount of a compound capable of reducing the activity of Core 2 GlcNAc-T. Preferably the compound will be used to reduce the activity of Core 2 GlcNAc-T to normal or approximately normal levels.

The activity of Core 2 GlcNAc-T can be reduced in a number of ways, for example by inhibiting the transcription of the Core 2 GlcNAc-T gene, by inhibiting the translation of the Core 2 GlcNAc-T mRNA, by inhibiting the post translational modification of the protein (e.g. by inhibiting the phosphorylation of the protein through protein kinase and thereby inhibiting its activation) or by inhibiting the enzyme activity.

Inhibitors of both Core 2 GlcNAc-T enzyme activity and of the activation of Core 2 GlcNAc-T by protein kinase Cβ are known. Conveniently the level of Core 2 GlcNAc-T enzyme activity is reduced either by inhibiting the enzyme or inhibiting the phosphorylation of the protein.

Examples of Core 2 GlcNAc-T inhibitors suitable for use in the invention are: βGal(1→3)α(6-deoxy)GalNAcα-Bn. (Hindsgaul et al (1991) J Biol Chem. 266(27):17858-62, Kuhns et al (1993) Glycoconjugate Journal 10, 381-394; the following compounds activated as described by Toki et al (1994) Biochem Biophys Res Commun. 198(2):417-23: Galβ1→3GalNAcα-pnp, Galβ1→3GalNAcα-onp GalNAcα-pnp GalNAcβ-pnp, GlcNAcβ-pnp, Galβ-pnp, GlcNAcβ1→3GalNAcα-pnp, L-Fucα1→2Galβ-pnp, GlcNAcα-pnp, Galβ1→3 GlcNAcβ-pn, Galβ1→6GlcNAcβ-pnp; steroidal glycosides described in applicants co pending WO05060977 (incorporated herein by reference), eg. Trigoneoside IVa, Glycoside F, Compound 3 (3β-26-(β-D-glucopyranosyloxy)-22-hydroxyfurost-5,25 (27)dien-3-yl O-6-deoxy-α-L-mannopyranosyl-(1→2)-O-[β-D-glucopyranosyl-(1→4)]-β-D-gluco-pyranoside), Pardarinoside C, Shatavarin I, Shatavarin IV, Deltonin, Balanitin VI, solasodine 3-O-α-L-rhamnopyranosyl-(1→2)-O-[β-D-glucopyranosyl-(1→4)]-β-D-glucopyranoside, solandine 3-O-α-L-rhamnopyranosyl-(1→2)-O-[β-D-glucopyranosyl-(1→4)]-β-D-glucopyranoside; and analogues of uridine diphosphate and uridine diphosphate-N-acetylglucosamine and peptides of the formula X—X¹—X²—X³—X⁴ as described in WO0185748 (incorporated herein by reference).

Preferably the steroidal glycoside core 2 GlcNAc-T inhibitor comprises a sugar-derived substituent. The term sugar-derived substituent means a saccharide, in which optionally one or more hydrogens and/or one or more hydroxyl groups have been replaced by —R, —OR, —SR, —NR wherein R is methyl, ethyl or propyl to form a derivative.

Saccharides include, but are not limited to, monosaccharides, disaccharides, trisaccharides, tetrasaccharides and polysaccharides.

Monosaccharides include, but are not limited to, arabinose, xylose, lyxose, ribose, glucose, mannose, galactose, allose, altrose, gulose, idose, talose, ribulose, xylulose, fructose, sorbose, tagatose, psicose, sedoheptulose, deoxyribose, fucose, rhamnose, 2-deoxy-glucose, quinovose, abequose, glucosamine, mannosamine, galactosamine, neuraminic acid, muramic acid, N-acetyl-glucosamine, N-acetyl-mannosamine, N-acetyl-galactosamine, N-acetylneuraminic acid, N-acetylmuramic acid, O-acetylneuraminic acid, N-glycolylneuraminic acid, fructuronic acid, tagat-uronic acid, glucuronic acid, mannuronic acid, galacturonic acid, iduronic acid, sialic acid and guluronic acid.

Preferably, the core 2 GlcNAc-T inhibitor comprises at least one sugar-derived substituent; more preferably, the core 2 GlcNAc-T inhibitor comprises at least two sugar-derived substituents.

Preferably, each sugar-derived substituent is independently a mono-, di-, tri- or tetrasaccharide; more preferably, each sugar-derived substituent is independently a mono- or trisaccharide.

Preferably, the core 2 GlcNAc-T inhibitor is a compound of the formula I

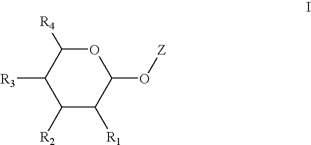

wherein R₁ is —OH, C₁₋₆ alkoxy, —NR₈R₉, or a monosaccharide of the formula: IIa:

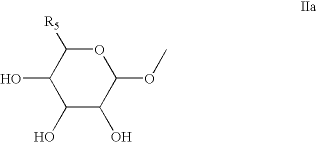

Preferably R₁ is —OH, —NR₈R₉, or a monosaccharide of the formula IIa; more preferably R₁ is —NR₈R₉, or a monosaccharide of the formula IIa; most preferably R₁ is a monosaccharide of the formula IIa;

R₂ is —OH, C₁₋₆ alkoxy or a monosaccharide of the formula IIb:

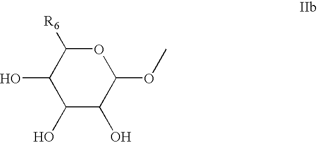

Preferably R₂ is —OH or a monosaccharide of the formula IIb; more preferably R₂ is —OH or a monosaccharide of the formula IIb; most preferably R₂ is —OH;

R₃ is —OH, C₁₋₆ alkoxy or a monosaccharide of the formula IIc:

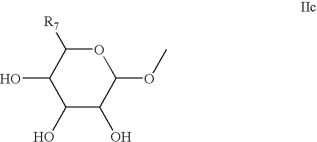

Preferably R₃ is —OH or a monosaccharide of the formula IIc; more preferably R₃ is a monosaccharide of the formula IIc; most preferably R₃ is glucose;

R₄ is C₁₋₆ alkyl, C₁₋₆ hydroxyalkyl or C₁₋₆-alkoxy-C₁₋₆-alkyl; preferably R₄ is C₁₋₆ alkyl or C₁₋₆ hydroxyalkyl; more preferably R₄ is —CH₂OH or —CH₃; most preferably R₄ is —CH₂OH;

R₅ is C₁₋₆ alkyl, C₁₋₆ hydroxyalkyl or C₁₋₆-alkoxy-C₁₋₆-alkyl; preferably R₅ is C₁₋₆ alkyl or C₁₋₆ hydroxyalkyl; more preferably R₅ is —CH₃, —C₂H₅, —CH₂OH or —C₂H₄OH; most preferably R₅ is —CH₃;

R₆ is C₁₋₆ alkyl, C₁₋₆ hydroxyalkyl or C₁₋₆-alkoxy-C₁₋₆-alkyl; preferably R₆ is C₁₋₆ alkyl or C₁₋₆ hydroxyalkyl more preferably R₆ is —CH₂OH or —CH₃; most preferably R₆ is —CH₂OH;

R₇ is C₂₋₆ alkyl, C₁₋₆ hydroxyalkyl or C₁₋₆-alkoxy-C₁₋₆-alkyl; preferably R₇ is C₁₋₆ hydroxyalkyl or C₁₋₆-alkoxy-C₁₋₆-alkyl; more preferably R₇ is —CH₂OH or C₁₋₆ alkoxymethyl; most preferably R₇ is —CH₂OH;

R₈ is H, C₁₋₆ alkyl or C₁₋₆ acyl; preferably R₈ is H or C₁₋₆ alkyl; more preferably R₈ is H or CH₃; most preferably R₈ is H;

R₉ is H, C₁₋₆ alkyl or C₁₋₆ acyl; preferably R₉ is H or C₁₋₆ acyl more preferably R₉ is H or —COCH₃; most preferably R₉ is —COCH₃; and

Z is a steroid group;

or a pharmaceutically acceptable salt, ester or tautomeric form or derivative thereof.

Preferably the compound of the formula I is a compound of the formula III:

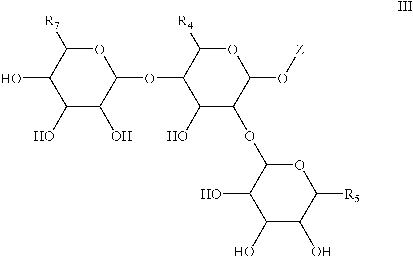

wherein:

R₄ is C₁₋₆ alkyl, C₁₋₆ hydroxyalkyl or C₁₋₆-alkoxy-C₁₋₆-alkyl; preferably C₁₋₆ alkyl or C₁₋₆ hydroxyalkyl more preferably —CH₂OH or —CH₃; most preferably —CH₂OH;

R₅ is C₁₋₆ alkyl, C₁₋₆ hydroxyalkyl or C₁₋₆-alkoxy-C₁₋₆-alkyl; preferably R₅ is C₁₋₆ alkyl or C₁₋₆ hydroxyalkyl; more preferably R₅ is —CH₃, —C₂H₅, —CH₂OH or —C₂H₄OH; most preferably R₅ is —CH₃; and

R₇ is C₂₋₆ alkyl, C₁₋₆ hydroxyalkyl or C₁₋₆-alkoxy-C₁₋₆-alkyl; preferably R₇ is C₁₋₆ hydroxyalkyl or C₁₋₆-alkoxy-C₁₋₆-alkyl; more preferably R₇ is —CH₂OH or C₁₋₆ alkoxymethyl; most preferably R₇ is —CH₂OH.

More preferred are compounds of the formula III wherein:

R₄ is C₁₋₆ hydroxyalkyl or C₁₋₆ alkyl;

R₅ is C₁₋₆ alkyl, C₁₋₆ hydroxyalkyl; and

R₇ is C₁₋₆ hydroxyalkyl or C₁₋₆-alkoxy-C₁₋₆-alkyl.

More preferred are compounds wherein:

R₄ is —CH₂OH or —CH₃;

R₅ is —CH₃; and

R₇ is —CH₃OH.

Most preferred compounds of the formula III are compounds of the formula I wherein:

R₁ is rhamnose;

R₂ is —OH;

R₃ is glucose; and

R₄ is —CH₂OH.

Most preferred are compounds of the formula I which are of the formula IV:

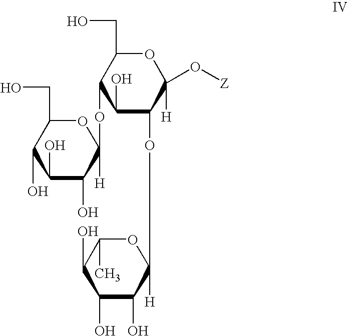

Also provided are compounds wherein the compound of the formula I is a compound of the formula V:

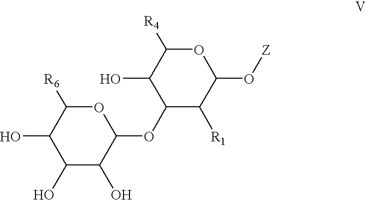

wherein:

R₁ is —OH, C₁₋₆ alkoxy or NR₈R₉, or a monosaccharide of the formula IIa:

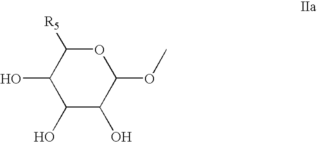

Preferably R₁ is —OH, or NR₈R₉; more preferably R₁ is NR₈R₉.

R₄ is C₁₋₆ alkyl, C₁₋₆ hydroxyalkyl or C₁₋₆-alkoxy-C₁₋₆-alkyl; preferably R₄ is C₁₋₆ alkyl or C₁₋₆ hydroxyalkyl more preferably R₄ is C₁₋₆ alkyl; most preferably —CH₃;

R₅ is C₁₋₆ alkyl, C₁₋₆ hydroxyalkyl or C₁₋₆-alkoxy-C₁₋₆-alkyl; preferably R₅ is C₁₋₆ alkyl or C₁₋₆ hydroxyalkyl; more preferably R₅ is —CH₃ or —CH₂OH; most preferably R₅ is —CH₃; and

R₆ is C₁₋₆ alkyl, C₁₋₆ hydroxyalkyl or C₁₋₆-alkoxy-C₁₋₆-alkyl; preferably R₆ is C₁₋₆ alkyl or C₁₋₆ hydroxyalkyl more preferably R₆ is —CH₂OH or —CH₃; most preferably R₆ is —CH₂OH;

R₈ is H, C₁₋₆ alkyl or C₁₋₆ acyl; preferably R₈ is H or C₁₋₆ alkyl; more preferably R₈ is H or CH₃; most preferably R₈ is H;

R₉ is H, C₁₋₆ alkyl or C₁₋₆ acyl; preferably R₉ is H or C₁₋₆ acyl more preferably R₉ is H or —COCH₃; most preferably R₉ is —COCH₃; and

Z is a steroid group.

Preferred compounds of the formula V are compounds in which:

R₁ is —OH, C₁₋₆ alkoxy or NR₈R₉;

R₄ is C₁₋₆ alkyl or C₁₋₆ hydroxyalkyl;

R₆ is C₁₋₆ alkyl or C₁₋₆ hydroxyalkyl;

R₈ is H, C₁₋₆ alkyl or C₁₋₆ acyl; and

R₉ is H, C₁₋₆ alkyl or C₁₋₆ acyl.

More preferred compounds of the formula IV are those in which:

R₁ is —NH—C₁₋₆-acyl;

R₄ is C₁₋₆ alkyl or —CH₂OH; and

R₆ is C₁₋₆ hydroxyalkyl.

Most preferred are the compounds of the formula IV which are of the formula: Galβ1→3(6-deoxy)GalNAcα-Z

The compounds of the formula I comprise a steroid group. The term “steroid group” means a group comprises the tetracyclic ring system shown as formula VI:

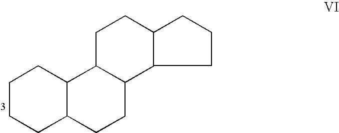

Preferably the steroid group is attached to the rest of the molecule through the 3-position of the steroid group. For example compounds of the formula I above are preferably compounds of the formula:

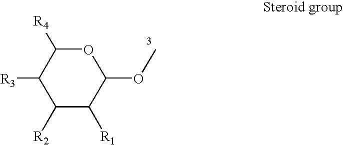

The steroid group may be cholestane, 5α-pregnane, androstane, estrane, cholesterol, cholane, a progestin, a glucocorticoid, a mineralocorticoid, an androgen such as dehydroepiandrosterone or its 7-keto analogue, a bile acid or other steroid. In one preferred embodiment the steroid core is a steroid that is in itself beneficial or neutral. By neutral is meant that the steroid itself has been passed suitable for use in a human or animal. By beneficial is meant that the steroid has effects of benefit to the human or animal if it were administered separately.

The steroid group may be a steroidal sapogenin derivable from plant sources or a steroidal sapogenin which is itself derivable from such plant steroidal sapogenins by chemical modification.

In one embodiment the steroid group is a steroidal sapogenin of the formula VII:

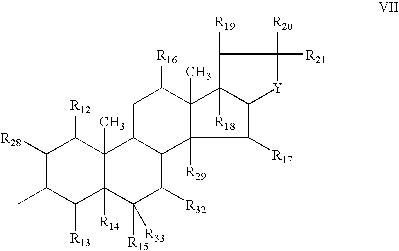

wherein:

R₁₂ is H, OH, C₁₋₆ alkyl or C₁₋₆ alkoxy; preferably R₁₂ is H or —OH; most preferably R₁₂ is H;

R₁₃ is H, —OH, ═O, or C₁₋₆ alkyl; preferably R₁₃ is H or —OH; most preferably R₁₃ is H;

R₁₄ is H, —OH or C₁₋₆ alkyl or R₁₄ and R₃₃ taken together represent the second bond of a double bond joining adjacent carbon atoms; preferably R₁₄ is H or R₁₄ and R₃₃ taken together represent the second bond of a double bond joining adjacent carbon atoms;

R₁₅ is H, or —OH, or R₁₅ and R₃₃ taken together are ═O; preferably R₁₅ is H, or R₁₅ and R₃₃ taken together are ═O; more preferably R₁₅ is H;

R₁₆ is H, OH or ═O; preferably R₁₆ is H or ═O; more preferably R₁₆ is H;

R₁₇ is H, OH or ═O; preferably R₁₇ is H or —OH; more preferably R₁₇ is H;

R₁₈ is H, OH, C₁₋₆ alkoxy or C₁₋₆ alkyl; preferably R₁₈ is H, OH, C₁₋₆ alkoxy; more preferably R₁₈ is H or OH; most preferably R₁₈ is H;

R₁₉ is H, OH, C₁₋₆ alkyl or C₁₋₆ alkoxy; preferably R₁₉ is H, OH, C₁₋₆ alkyl; more preferably R₁₉ is H, OH or C₁₋₆ alkyl; most preferably R₁₉ is C₁₋₆ alkyl; and particularly R₁₉ is —CH₃;

R₂₀ is H, OH, C₁₋₆ alkoxy or C₁₋₆ alkyl; preferably R₂₀ is H, —OH, or C₁₋₆ alkoxy; more preferably R₂₀ is —OH or C₁₋₆ alkoxy; most preferably R₂₀ is —OH;

R₂₁ is H, OH, C₁₋₆ alkyl, C₁₋₆ alkoxy or is a group of the formula VIII:

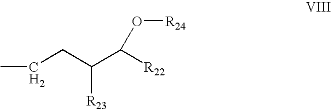

preferably R₂₁ is a group of the formula VIII;

R₂₂ is H, OH, C₁₋₆ alkyl or C₁₋₆ alkoxy; preferably R₂₂ is H, OH, or C₁₋₆ alkoxy; preferably R₂₂ is H or OH, —OCH₃ or —O—C₂H₅; most preferably R₂₂ is H;

R₂₃ is H, OH, C₁₋₆ alkyl, C₁₋₆ hydroxyalkyl, C₁₋₆-alkoxy-C₁₋₆-alkyl, ═CH₂ or ═CH—C₁₋₆-alkyl; preferably R₂₃ is C₁₋₆ alkyl, C₁₋₆ hydroxyalkyl, C₁₋₆-alkoxy-C₁₋₆-alkyl, ═CH₂ or ═CH—C₁₋₆-alkyl; more preferably R₂₃ is C₁₋₆ alkyl, C₁₋₆ hydroxyalkyl or ═CH₂; most preferably R₂₃ is —C₂H₄OH, —CH₂OH, C₁₋₆ alkyl, or ═CH₂, even more preferably R₂₃ is —C₂H₄OH, —CH₂OH, —C₂H₅, —CH₃ or ═CH₂ and particularly R₂₃ is —CH₃ or ═CH₂; and

R₂₄ is H, C₁₋₆ alkyl, C₁₋₆ acyl or a monosaccharide MS; preferably R₂₄ is C₁₋₆ alkyl, C₁₋₆ acyl or a monosaccharide MS; more preferably R₂₄ is C₁₋₆ acyl or a monosaccharide MS; most preferably R₂₄ is a monosaccharide MS.

R₂₈ and R₂₉ are the same or different and are H or OH; preferably R₂₈ is H and R₂₉ is —OH; more preferably both R₂₈ and R₂₉ are H;

R₃₂ is H, OH or ═O; preferably R₃₂ is H or OH; most preferably R₃₂ is H; and

R₃₃ is H, or R₃₃ and R₁₅ taken together are ═O, or R₃₃ and R₁₄ taken together represent the second bond of a double bond joining adjacent carbon atoms; preferably R₃₃ is H or R₃₃ and R₁₄ taken together represent the second bond of a double bond joining adjacent carbon atoms;

MS is selected from a group consisting of arabinose, xylose, lyxose, ribose, glucose, mannose, galactose, allose, altrose, gulose, idose, talose, ribulose, xylulose, fructose, sorbose, tagatose, psicose, sedoheptulose, deoxyribose, fucose, rhamnose, 2-deoxy-glucose, quinovose, abequose, glucosamine, mannosamine, galactosamine, neuraminic acid, muramic acid, N-acetyl-glucosamine, N-acetyl-mannosamine, N-acetyl-galactosamine, N-acetylneuraminic acid, N-acetylmuramic acid, O-acetylneuraminic acid, N-glycolylneuraminic acid, fructuronic acid, tagaturonic acid, glucuronic acid, mannuronic acid, galacturonic acid, iduronic acid, sialic acid and guluronic acid; preferably MS is selected from a group consisting of glucose, galactose, mannose, fucose, N-acetyl-glucosamine, N-acetyl-galactosamine and sialic acid; most preferably MS is glucose; and

Y is N or O; preferably Y is O.

Preferred steroidal sapogenins of the formula VII are those in which R₂₁ is of the formula VIII and Y is O.

More preferred steroidal sapogenins of the formula VII are those in which:

R₁₂ is H, —OH

R₁₃ is H or —OH;

R₁₄ is H, or —OH or R₁₄ and R₃₃ taken together represent the second bond of a double bond joining adjacent carbon atoms;

R₁₅ is H, or R₁₅ and R₃₃ taken together are ═O;

R₁₈ is H, —OH or C₁₋₆ alkoxy

R₁₉ is C₁₋₆ alkyl;

R₂₀ is H, —OH or C₁₋₆ alkoxy;

R₂₈ is H;

R₃₂ is H, —OH or ═O; and

R₃₃ is H, or R₃₃ and R₁₅ taken together are ═O, or R₃₃ and R₁₄ taken together represent the second bond of a double bond joining adjacent carbon atoms.

Most preferred are steroidal sapogenins of the formula VII in which:

R₁₂, R₁₃, R₁₅ and R₂₈ each represent H;

R₁₄ is H, or R₁₄ and R₃₃ taken together represent the second bond of a double bond joining adjacent carbon atoms;

R₁₆ is H, or ═O;

R₁₇ is H or —OH;

R₁₈ is H or —OH;

R₁₉ is H, or C₁₋₆ alkyl;

R₂₁ is of the formula VIII;

R₂₂ is H, —OH, or C₁₋₆ alkoxy;

R₂₄ is C₁₋₆ alkyl, C₁₋₆ acyl, or glucose;

R₂₉ is H or —OH; and

R₃₂ is H or —OH.

The most preferred steroidal sapogenins of the formula VII are those in which

R₁₂, R₁₃, R₁₅, R₁₆, R₁₇, R₂₂, R₂₈ each represent H;

R₁₄ is H, or R₁₄ and R₃₃ taken together represent the second bond of a double bond joining adjacent carbon atoms;

R₂₀ is —OH or C₁₋₆ alkoxy;

R₂₁ is of the formula VIII;

R₂₃ is —CH₃ or ═CH₂;

R₂₄ is C₁₋₆ acyl or glucose;

R₂₉ is H or —OH; and

R₃₂ is H.

The most preferred steroidal sapogenins of the formula VII are selected from the group consisting of:

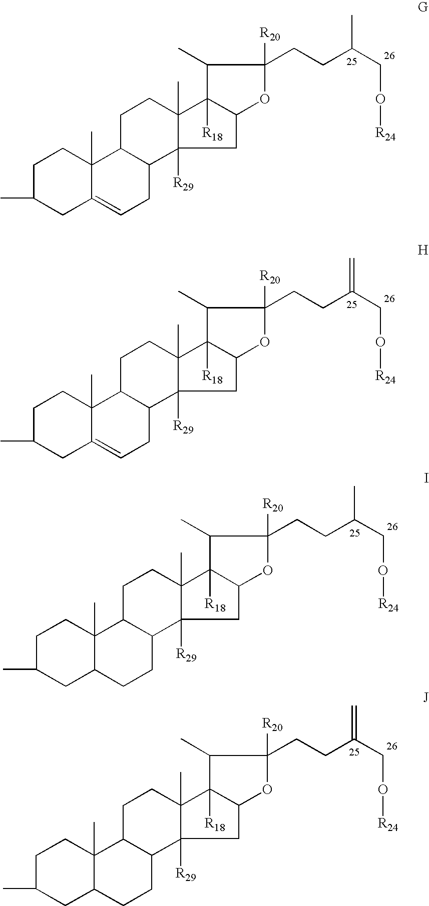

wherein:

R₁₈ is H or OH;

R₂₀ is OH or C₁₋₆ alkoxy;

R₂₄ is glucose or C₁₋₆ acyl; and

R₂₉ is H or OH.

Particularly preferred compounds of the formula I in which the steroid group is of the formula VII are trigoneoside IVa, glycoside F, shatavarin I, compound 3, pardarinoside C, whose structures are summarised in Table 1.

TABLE 1
Structural details of trigoneoside IVa, glycoside F, shatavarin I,
compound 3 and pardarinoside C

| | | Steroid | | | | | | |
|---|---|---|---|---|---|---|---|---|
| Compound | Ref. | group | R₁₈ | R₂₀ | R₂₉ | C²⁵ | R/S | C²⁶ |
| Trigoneoside IVa | 55 | G | H | —OH | H | —CH₃ | S | Glc |
| Glycoside F | 55 | G | H | —OH | H | —CH₃ | R | Glc |
| Shatavarin I | 56 | I | H | —OH | H | —CH₃ | S | Glc |
| Compound 3 | This | H | H | —OH | H | ═CH₂ | ? | Glc |
| | document | | | | | | | |
| Pardarinoside C | 57 | I | OH | —OMe | —OH | —CH₃ | R | acetyl |

In each case the saccharide group bonded to the steroid group at the 3-position is: 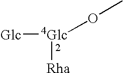
Alternatively the steroid group may be a steroidal sapogenin of the formula VIII:

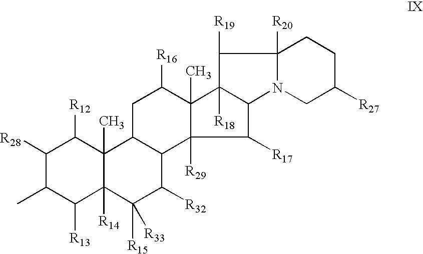

wherein:

R₁₂ is H, —OH, C₁₋₆ alkyl or C₁₋₆ alkoxy; preferably R₁₂ is H or —OH; most preferably R₁₂ is H;

R₁₃ is H, —OH, ═O, or C₁₋₆ alkyl; preferably R₁₃ is H or —OH; most preferably R₁₃ is H;

R₁₄ is H—OH or C₁₋₆ alkyl or R₁₄ and R₃₃ taken together represent the second bond of a double bond joining adjacent carbon atoms; preferably R₁₄ is H or R₁₄ and R₃₃ taken together represent the second bond of a double bond joining adjacent carbon atoms;

R₁₅ is H, or —OH, or R₁₅ and R₃₃ taken together are ═O; preferably R₁₅ is H, or R₁₅ and R₃₃ taken together are ═O; more preferably R₁₅ is H;

R₁₆ is H, —OH or ═O; preferably R₁₆ is H or ═O; more preferably R₁₆ is H;

R₁₇ is H, —OH or ═O; preferably R₁₇ is H or —OH; more preferably R₁₇ is H;

R₁₈ is H, —OH, C₁₋₆ alkoxy or C₁₋₆ alkyl; preferably R₁₈ is H, —OH, C₁₋₆ alkoxy; more preferably R₁₈ is H or OH; most preferably R₁₈ is H;

R₁₉ is H, —OH, C₁₋₆ alkyl or C₁₋₆ alkoxy; preferably R₁₉ is H, OH, or C₁₋₆ alkyl; more preferably R₁₉ is C₁₋₆ alkyl; and particularly R₁₉ is —CH₃;

R₂₀ is H, —OH, C₁₋₆ alkoxy or C₁₋₆ alkyl; preferably R₂₀ is H, —OH, or C₁₋₆ alkoxy; more preferably R₂₀ is —OH or C₁₋₆ alkoxy; most preferably R₂₀ is —OH;

R₂₇ is H, —OH, C₁₋₆ alkyl, C₁₋₆ alkoxy or C₁₋₆ hydroxyalkyl; preferably R₂₇ is H, C₁₋₆ alkyl or C₁₋₆ alkoxy; more preferably R₂₇ is H or C₁₋₆ alkyl; most preferably R₂₇ is methyl, ethyl or propyl;

R₂₈ and R₂₉ are the same or different and are H or —OH; preferably both R₂₈ and R₂₉ are H;

R₃₂ is H, —OH or ═O; preferably R₃₂ is H or —OH; most preferably R₃₂ is H; and

R₃₃ is H, or R₃₃ and R₁₅ taken together are ═O, or R₃₃ and R₁₄ taken together represent the second bond of a double bond joining adjacent carbon atoms; preferably R₃₃ is H or R₃₃ and R₁₄ taken together represent the second bond of a double bond joining adjacent carbon atoms.

Preferred steroidal sapogenins of the formula IX are those in which:

R₁₂ is H or —OH

R₁₃ is H or —OH;

R₁₄ is H or —OH, or R₁₄ and R₃₃ taken together represent the second bond of a double bond joining adjacent carbon atoms;

R₁₅ is H or —OH

R₁₆ is H, —OH or ═O;

R₁₇ is H, —OH or ═O;

R₁₈ is H or —OH

R₂₇ is C₁₋₆ alkyl; and

R₂₈ and R₂₉ are the same or different and each represent H or —OH;

R₃₂ is H, —OH or ═O.

More preferably steroidal sapogenins of the formula IX are those in which:

R₁₂ is H or —OH

R₁₃ is H or —OH;

R₁₄ is H or —OH, or R₁₄ and R₃₃ taken together represent the second bond of a double bond joining adjacent carbon atoms;

R₁₅ is H or —OH

R₁₆ is H or ═O;

R₁₇ is H, —OH;

R₁₈ is H or —OH;

R₂₇ is C₁₋₆ alkyl;

R₂₈ and R₂₉ are the same or different and each represent H or —OH; and

R₃₂ is H or —OH.

More preferably steroidal sapogenins of the formula IX are those in of the general formula IXa:

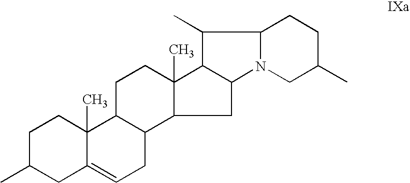

The most preferred compound of the formula I in which the steroid group is of the formula IX is: 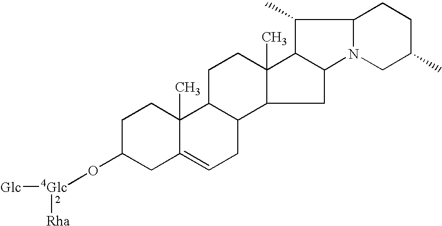
isolatable from Lilium macklineae (59).

A further preferred group of steroidal sapogenins are those in which the steroidal sapogenin is of the formula XI:

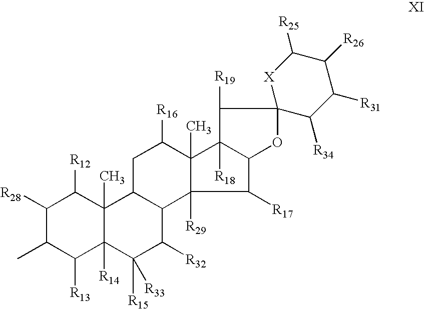

wherein:

R₁₂ is H, OH, C₁₋₆ alkyl or C₁₋₆ alkoxy; preferably R₁₂ is H or —OH; most preferably R₁₂ is H; —

R₁₃ is H, —OH, ═O, or C₁₋₆ alkyl; preferably R₁₃ is H or —OH; most preferably R₁₃ is H; —

R₁₄ is H, —OH or C₁₋₆ alkyl or R₁₄ and R₃₃ taken together represent the second bond of a double bond joining adjacent carbon atoms; preferably R₁₄ is H or R₁₄ and R₃₃ taken together represent the second bond of a double bond joining adjacent carbon atoms;—

R₁₅ is H, or —OH, or R₁₅ and R₃₃ taken together are ═O; preferably R₁₅ is H, or R₁₅ and R₃₃ taken together are ═O; more preferably R₁₅ is H; —

R₁₆ is H, —OH or ═O; preferably R₁₆ is H or ═O; more preferably R₁₆ is H;

R₁₇ is H, —OH or ═O; preferably R₁₇ is H or —OH; more preferably R₁₇ is H;

R₁₈ is H, —OH, C₁₋₆ alkoxy or C₁₋₆ alkyl; preferably R₁₈ is H, OH, C₁₋₆ alkoxy; more preferably R₁₈ is H or —OH; most preferably R₁₈ is H;

R₁₉ is H, —OH, C₁₋₆ alkyl or C₁₋₆ alkoxy; preferably R₁₉ is H, —OH, C₁₋₆ alkyl; more preferably R₁₉ is H, —OH or C₁₋₆ alkyl; most preferably R₁₉ is C₁₋₆ alkyl; and particularly R₁₉ is —CH₃;

R₂₅ is H, —OH, C₁₋₆ alkyl or C₁₋₆ alkoxy; preferably R₂₅ is H or —OH; more preferably R₂₅ is H;

R₂₆ is H, —OH, C₁₋₆ alkyl, C₁₋₆ hydroxyalkyl, C₁₋₆-alkoxy-C₁₋₆-alkyl, ═CH₂ or ═CH—C₁₋₆-alkyl; preferably R₂₆ is C₁₋₆ alkyl, C₁₋₆ hydroxyalkyl, C₁₋₆-alkoxy-C₁₋₆-alkyl, ═CH₂ or ═CHCl₆ alkyl; more preferably R₂₆ is C₁₋₆ alkyl, C₁₋₆ hydroxyalkyl or ═CH₂; most preferably R₂₆ is —C₂H₄OH, —CH₂OH, C₁₋₆ alkyl, or ═CH₂, even more preferably R₂₆ is —C₂H₄OH, —CH₂OH, —C₂H₅, —CH₃ or ═CH₂ and particularly R₂₆ is CH₃ or ═CH₂;

R₂₈ and R₂₉ are the same or different and are H or —OH; preferably both R₂₈ and R₂₉ are H;

R₃₁ is H or —OH; preferably R₃₁ is H;

R₃₂ is H, —OH or ═O; preferably R₃₂ is H or —OH; most preferably R₃₂ is H;

R₃₃ is H, or R₃₃ and R₁₅ taken together are ═O, or R₃₃ and R₁₄ taken together represent the second bond of a double bond joining adjacent carbon atoms; preferably R₃₃ is H or R₃₃ and R₁₄ taken together represent the second bond of a double bond joining adjacent carbon atoms;

R₃₄ is H or —OH; preferably R₃₄ is H; and

X is O, S or NH; preferably X is O or NH; more preferably X is O.

Preferred steroidal sapogenins of the formula XI are those in which:

R₁₂ is H or —OH;

R₁₃ is H or —OH;

R₁₄ is H or —OH, or R₁₄ and R₃₃ taken together represent the second bond of a double bond joining adjacent carbon atoms;

R₁₅, R₁₈ R₂₈ and R₂₉ are the same or different and each represent H or —OH,

R₁₆ is H, OH or ═O;

R₁₇ is H, —OH or ═O;

R₁₈ is H, —OH or C₁₋₆-alkoxy;

R₁₉ is H, or C₁₋₆ alkyl;

R₂₆ is H, C₁₋₆ alkyl, C₁₋₆ hydroxyalkyl, C₁₋₆-alkoxy-C₁₋₆-alkyl, ═CH₂ or ═CH—C₁₋₆-alkyl;

R₂₉ is H or —OH;

R₃₁ is H or —OH;

R₃₂ is H, —OH or ═O; and

R₃₃ is H, or R₃₃ and R₁₅ taken together are ═O, or R₃₃ and R₁₄ taken together represent the second bond of a double bond joining adjacent carbon atoms; and

R₃₄ is H or —OH.

More preferred steroidal sapogenins of the formula XI are those in which:

R₁₂, R₁₃, R₁₅ and R₂₈ each represent H;

R₁₄ is H, or R₁₄ and R₃₃ taken together represent the second bond of a double bond joining adjacent carbon atoms;

R₁₆ is H, or ═O;

R₁₇ is H or —OH;

R₁₈ is H or —OH;

R₁₉ is H, or C₁₋₆ alkyl;

R₂₆ is C₁₋₆ alkyl, C₁₋₆ hydroxyalkyl or ═CH₂;

R₂₈ is H;

R₂₉ is H or —OH;

R₃₂ is H or —OH; and

R₃₃ is H, or R₃₃ and R₁₄ taken together represent the second bond of a double bond joining adjacent carbon atoms.

Most preferred steroidal sapogenins of the formula XI are those in which:

R₁₂, R₁₃, R₁₅, R₁₆, R₁₇, R₂₅, R₂₈, R₃₁, R₃₂ and R₃₄, each represent H;

R₁₄ is H, or R₁₄ and R₃₃ taken together represent the second bond of a double bond joining adjacent carbon atoms;

R₁₈ is H or —OH;

R₁₉ is C₁₋₆ alkyl;

R₂₆ is C₁₋₆ alkyl or ═CH₂;

R₂₉ is H or —OH;

R₃₂ is H;

R₃₃ is H, or R₃₃ and R₁₄ taken together represent the second bond of a double bond joining adjacent carbon atoms.

The most preferred steroidal sapogenins of the formula XI are those selected from the groups:

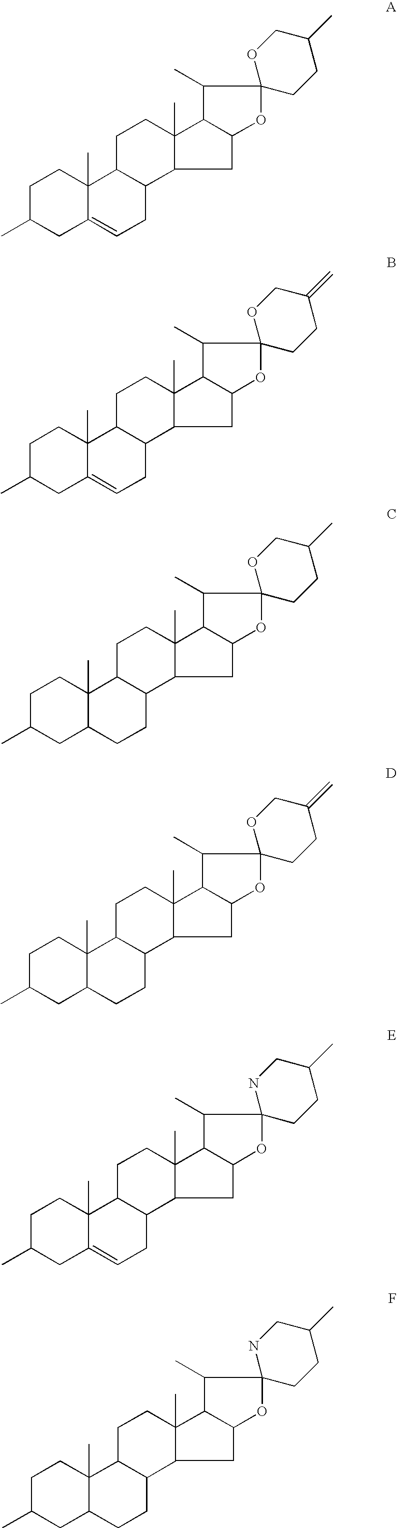

Particularly preferred steroidal sapogenins of the formula XI are diosgenin, yamogenin, tigogenin, neotigogenin, sarsasapogenin, smilagenin, hecogenin, solasodine or tomatidine.

Particularly preferred compounds of the formula I in which the steroidal group is of the formula XI are:

Shatavarin IV, (25R)shatavarin IV, deltonin, balanitin VI, compound 12 of Mimaki and Sahida (58).

Shatavarin IV is sarsasapogenin 3-O-α-L-rhamnopyranosyl-(1→2)-O-[β-D-glucopyranosyl-(1→4)]-β-D-glucopyranoside

Compound 12 is solasodine 3-O-α-L-rhamnopyranosyl-(1->2)-O-[β-D-glucopyranosyl-(1→4)]-β-D-glucopyranoside

Deltonin is (3β,25R)-spirost-5-en-3-yl-O-α-L-rhamnopyranosyl-(1→2)-O-[β-D-glucopyranosyl-β-D-Glucopyranoside.

Balanitin VI is (3β,25S)-spirost-5-en-3-yl-O-α-L-rhamnopyranosyl-(1→2)-O-[β-D-glucopyranosyl-β-D-Glucopyranoside.

Particularly preferred compounds of the formula I are those combining preferred steroid groups with preferred saccharide groups.

The shorthand annotation: 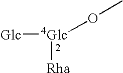
used in structures herein is used to denote the structure:

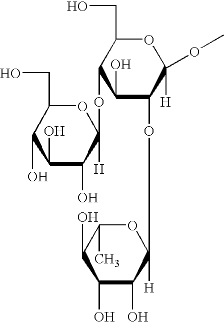

The short hand annotation:

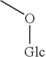

used in structures herein denotes the structure:

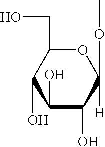

As used herein the shorthand annotation Glc is glucose and Rha is rhamnose.

For the avoidance of doubt the term C₁₋₆ acyl is —CO—C₁₋₅-alkyl.

Further Core 2 GIcNAc-T inhibitors suitable for use in the invention are described in applicants co-pending applications GB051888.8 and GB0513881.3 (both incorporated herein by reference)

Antibodies to Core 2 GlcNAc-T may also be used to reduce the activity of the enzyme and suitable examples are described in Li et al (1999) Glycoconjugate Journal 16, 555-562 (1999), U.S. Pat. No. 5,684,134 and WO09043662) all of which are incorporated herein by reference.

Inhibitors of Protein Kinase-Cβ2 (PKCβ2) are known to inhibit Core 2 GlcNAc-T activation in diabetic complications, where Core 2 activity is known to be raised, (Chibber et al (2003) Diabetes. 52(6): 1519-27—incorporated herein by reference) and are known to inhibit leukocyte binding to epithelial cells in vitro. Examples of 3,4-di-indoyl-pyrrol-2,5-dione derivatives that inhibit PKCβ are found in, for example WO9535294 and WO9517182 both of which are incorporated herein by reference. A particular example of a PKCβ2 inhibitor is Ruboxistaurin (LY333531 & LY379196).

In a second aspect of the invention is provided the use of compound capable of reducing Core 2 GlcNAc-T activity in the manufacture of a medicament for the treatment of Multiple Sclerosis. Examples of such compounds are as described above in the first aspect of the invention. For example such compounds are either inhibitors of Core 2 GIcNAc-T or inhibitors of PKCβ (especially of PKCβ2); preferably compounds are inhibitors of Core 2 GlcNAc-T.

In a third aspect of the invention is provided a method of diagnosing Multiple Sclerosis in a subject comprising comparing the level of Core 2 GlcNAc-T activity associated with leukocytes of a subject with the level of Core 2 GlcNAc-T activity determined in healthy non afflicted individuals. A level of Core 2 GlcNAc-T higher than that of healthy non afflicted individuals being indicative that the subject is afflicted with MS.

The measurement of Core 2 GlcNAc-T activity is preferably carried out on isolated tissue samples, such as biopsy samples or blood samples. Conveniently the measurement will be carried out by assay of Core 2 GlcNAc-T from isolated blood cells and particularly on preparations containing leukocytes, preferably substantially free of red blood cells. One such suitable procedure using leukocytes isolated from blood samples is described in Chibber et al Diabetes 49, 1724-1730 (2000). Typically values of Core 2 GlcNAc-T activity associated with leukocytes of a subject will be compared to an established normal level for healthy non afflicted individuals.

The inventors have determined that the level of Core 2 GlcNAc-T activity in leukocyte preparations obtained from healthy individuals and assayed by the method of Chibber et al (2000) id or as detailed in Example 1 is between 40 and 1000 pmoles/hr/mg (oligosaccharide incorporated per mg protein) and typically between 50 and 500 pmoles/hr/mg of protein values obtained for three groups of healthy control individuals were 249±35 9 (n=25), 334±86 (n=11) and 283±37 (n=31) pmols/hr/mg.

Levels of Core 2 GlcNAc-T in individuals afflicted with MS have been noted to be in the region of at least 2 times, for example at least 4 times, at least 6 times and most typically at least 8 times the level of healthy non afflicted individuals when leukocytes from blood samples assayed according to the above methods.

In a fourth aspect of the invention is provided a method of determining the utility of a test substance as useful in the treatment of MS comprising determining the ability of the substance to inhibit the activity of Core 2 GlcNAc-T, particularly that activity associated with leukocytes.

Conveniently inhibition of Core 2 GlcNAc-T activity can be determined by comparing the level of Core 2 GlcNAc-T activity obtained in an assay in which a test substance is incorporated to the level of Core 2 GlcNAc-T activity in the assay with no test substance.

Conveniently inhibition of Core 2 GlcNAc-T enzyme activity can be determined by a method comprising (a) contacting source of active Core 2 GlcNAc-T enzyme with an acceptor and a sugar donor for a Core 2 GlcNAc-T in the presence and absence of the test substance; (b) measuring the amount of sugar donor transferred to the acceptor, and relating decreased transfer in presence of test substance as compared to that in its absence to Core 2 GlcNAc-T inhibitory activity. It is particularly preferred and convenient to measure such activity on Core 2 GlcNAc-T present in or derived from leukocytes, particularly of an MS patient.

Any source of Core 2 GlcNAc-T activity may be used, for example an enzyme produced by recombinant means such as those disclosed in WO04111196 or U.S. Pat. No. 5,658,778 (incorporated herein by reference) or a tissue or cell culture or a preparation exhibiting measurable Core 2 GlcNAc-T activity derivable therefrom, for example U937 cells or heart lysates as described in applicants co pending application PCT GB/2004/005398 (incorporated by reference).

Examples of sugar donors and acceptors and the general conditions for assaying Core 2 GlcNAc-T activity are well known in the art e.g. Chibber et al (2000) id Hindsgaul et al (1991) id, Kuhns et al (1993) id Toki et al (1994) id and Orlacchio et al (1997) id (all of which are incorporated herein by reference). Such methods can be adapted for use in the fourth aspect of the invention by incorporation of test substances as described above. Further examples of assays according to the invention are given in WO 0031109 and in applicants co-pending application PCT GB/2004/005398. Conveniently the sugar donor is UDP-GlcNAc and the sugar acceptor is βGal(1-3)DαGalNAc-p-nitrophenol.

The term treating MS, as used herein, includes treating as prophylaxis and the treatment of existing disease. MS includes for example relapsing/remitting, secondary progressive, progressive relapsing and primary progressive forms of the condition. Other forms include benign, malignant, chronic/progressive and transitional/progressive Multiple Sclerosis.

Medicaments comprising compounds that reduce the activity of Core 2 GlcNAc-T, unless in raw foodstuff form, will preferably be provided sterile and or pyrogen free. Particularly aqueous medicaments such as aqueous solutions or suspensions and especially those for use in parenteral applications, will be made up in sterile and pyrogen free water. Medicaments of the second embodiment comprising the compounds of the invention can be administered by oral or parenteral routes, including intravenous, intramuscular, intraperitoneal, subcutaneous, transdermal, airway (aerosol), rectal, vaginal and topical (including buccal and sublingual) administration. For oral administration, the compounds of the invention will generally be provided in the form of tablets or capsules, as a powder or granules, or as an aqueous solution or suspension.

Tablets for oral use may include the active ingredients mixed with pharmaceutically acceptable excipients such as inert diluents, disintegrating agents, binding agents, lubricating agents, sweetening agents, flavouring agents, colouring agents and preservatives. Examples of suitable inert diluents include sodium and calcium carbonate, sodium and calcium phosphate, and lactose, while corn starch and alginic acid are examples of suitable disintegrating agents. Binding agents include, for example starch and gelatine, while the lubricating agent, if present, may for example, be magnesium stearate, stearic acid or talc. If desired, the tablets may be coated with an enteric coating material, such as glyceryl mono stearate or glyceryl distearate, to delay absorption in the gastrointestinal tract. Capsules for oral use include hard gelatine capsules in which the active ingredient is mixed with a solid diluent, and soft gelatine capsules wherein the active ingredients is mixed with water or an oil such as peanut oil, liquid paraffin or olive oil

Formulations for rectal administration may for example be presented as a suppository with a suitable base comprising, for example, cocoa butter or a salicylate.

Formulations suitable for vaginal administration may for example be presented as pessaries, tampons, creams, gels, pastes, foams or spray formulations containing in addition to the active ingredient such carriers as are known in the art to be appropriate.

For intramuscular, intraperitoneal, subcutaneous and intravenous use, the compounds of the invention will generally be provided in sterile solutions or suspensions, buffered to an appropriate pH and isotonicity. For example suitable aqueous vehicles include Ringer's solution and isotonic sodium chloride. Aqueous suspensions according to the invention may include suspending agents such as cellulose derivatives, sodium alginate, polyvinylpyrrolidone and gum tragacanth, and a wetting agent such as lecithin. Liposome formulations may also be used. Suitable preservatives include ethyl and n-propyl p-hydroxybenzoate.

The Core 2 GlcNAc-T inhibitors of the invention may also be incorporated to a food or beverage product.

In general a suitable dose of Core 2 GlcNAc-T inhibitor will be in the range of 10 ng to 50 mg per kilogram body weight of the recipient per day, preferably in the range of 100 ng to 10 mg, more preferably 1 μg to 1.0 mg/kg/d. The desired dose is preferably presented once daily. These sub-doses may be administered in unit dosage forms, for example, containing 10 μg to 1500 mg, preferably 100 μg to 1000 mg, and most preferably 1 mg to 700 mg of active ingredient per unit dosage form.

The present invention will now be described further by reference to the following non-limiting Examples, Schemes and Figures. Further embodiments falling within the scope of the invention will occur to those skilled in the art in the light of these

### FIGURES

FIG. 1 is a graph illustrating the levels of Core 2 GlcNAc-T activity in leukocytes from healthy control individuals and subjects with newly diagnosed MS.

FIG. 2 is a graph illustrating the data of FIG. 1 as a scatter plot.

FIGS. 3a and 3b are graphs illustrating the effect of purified trigoneoside IVa, glycoside F, and shatavarin IV on Core 2 GlcNAc-T activity in cell free (FIG. 3a) and cell based (FIG. 3b) assays. Test compounds were used at a final concentration of 20 ng/ml. TIVa=Trigoneoside IVa, Gly-F=Glycoside F, SHIV=Shatavarin IV.

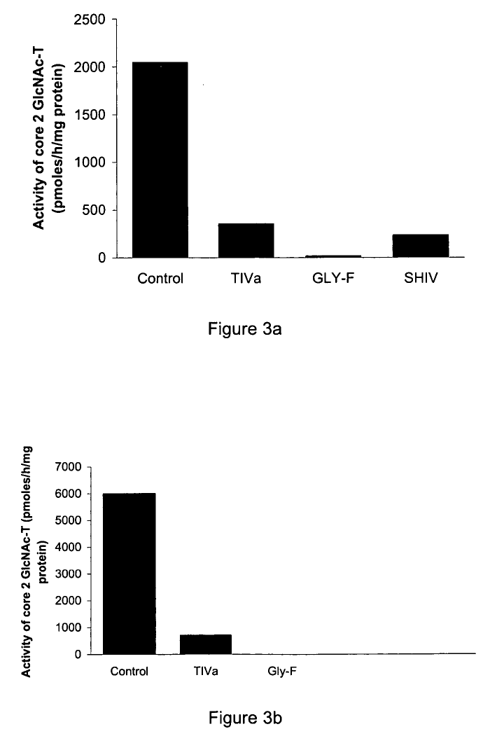

### EXAMPLES

### Example 1

### Determination of Core 2 GlcNAc-T Activity in Leukocytes Isolated from Patients Newly Diagnosed with MS

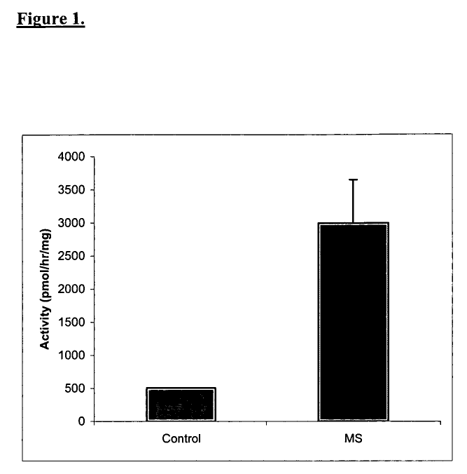

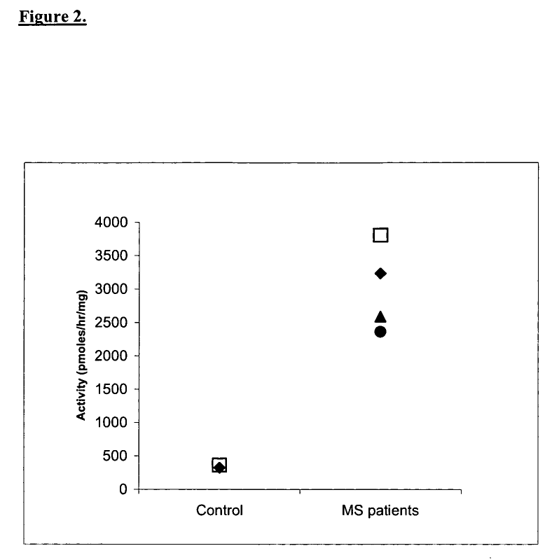

Blood samples were taken from 4 patients newly diagnosed with active MS and 2 age matched healthy control subjects and placed in heparinised tubes. The blood sample was layered onto an equal volume of Histo-Paque 1077™ (Sigma, Poole, Dorset, UK)”. and centrifuged at 400 g for 30 mins. The Buffy coat (containing peripheral blood mononuclear cells (PBMNC) and polymorphonuclear (PMN) leukocytes) was washed in phosphate buffered saline. Isolated leukocytes were frozen and lysed in 0.9% NaCl 0.4% Triton-X100 1 mM PMSF and the Core 2 GlcNAc-T assayed. The reaction was performed in 50 mmol/l 2(N-morpholino) 2(N-morpholino) ethanesulfonic acid pH 7.0; 1 mmol/l UDP GlcNAc, 0.5 μCi UDP-6 [3H]-N-acetylglucosamine (16,000 dpm/nmol, NEN Life Science Products, Hounslow, U.K.); 0.1 mol/l GlcNAc; 1 mmol/l βDgal (1-3)Dα-GalNAc-p-nitrophenol and 15 μl cell lysate (100-200 μg protein) for a final volume of 30 μl. After incubating the mixture for 1 h at 37° C., the reaction was terminated by adding 1 ml of ice cold water and processed on a C18 Sep-Pak column (Waters-Millipore, Watford, U.K.). After washing the column with 20 ml water, the product was eluted with 5 ml methanol and radioactivity counted. Endogenous activity of core 2 transferase was measured in the absence of the added acceptor. The results are shown in FIGS. 1 and 2.

2. Inhibition of Core 2 GlcNAc-T Activity by Inhibitors of Core 2 GlcNAc-T

2a Cell Based Assay

Human leukocytes (U937 cells) were exposed to human recombinant TNF-alpha (8 pg/ml) in the presence and absence of test compounds After 24 h incubation, the activity of Core 2 GlcNAc-T was measured as above.

2b Cell Free Assay

In cell free assays of Core 2 GlcNAc-T Heart lysates from either from TNF-alpha over expressing transgenic mice (female, B6.5JL-Tg (TNF) supplied by Taconic-M+B, Bomholtveg 10, 8680 Ry, Denmark) or from BB rats (Festing M.F.W. (Ed.). Inbred strains in biomedical research. The Macmillan Press Ltd, London (1979). ISBN 0-333-23809-5) were exposed to various concentrations of test compound for 1 h at 37° C. Activity of Core 2 GlcNAc-T was measured as above.

Trigoneoside IVa and Glycoside F purified from fenugreek seeds (Yoshikawa et al 1998 Heterocycles 47, 397-405) and Shatavarin IV (Ravikumar et al 1987 Indian J. Chem. 26B, 1012-1017, Joshi and Dev 1988 Indian J. Chem. 27B, 12-16) were tested as inhibitors in the above assays.

TABLE 2
Approximate IC₅₀ values (nM) for Core 2 GlcNAc-T inhibitors

| | Cell free | Cell based |
|---|---|---|
| Compound | assay | assay |
| Trigoneoside IVa | 0.9* | 0.75 |
| Glycoside F | 5** | a |
| Shatavarin IV | b | c |

*Cell free assays were carried out on heart lysates of TNF-α mice
**Cell free assays were carried out on heart lysates of BB rats
a 100% inhibition at 22 nM in BB rat heart lysate
b 89% inhibition at 22 nM in BB rat heart lysate
c no activity detected at 22.5 nM

1. A method of treatment of Multiple Sclerosis in a subject comprising administering to a subject in need thereof a therapeutically effective amount of a compound capable of reducing the activity of Core 2 GlcNAc-T.

2. The method of claim 1 wherein the substance capable of reducing the activity of Core 2 GlcNAc-T is an inhibitor of Core 2 GlcNAc-T enzyme activity.

3. The method of claim 1 in which the inhibitor of Core 2 GlcNAc-T activity is selected from the group consisting of steroidal glycosides, analogues of Uridine Diphosphate-N-Acetylglucosamine, analogues of Uridine Diphosphate, βGal(1→3)α(6-deoxy)GalNAcα-Bn and those compounds obtainable by UV activation of a compounds selected from the group consisting of Galβ1→3GalNAcα-pnp, Galβ1→3GalNAcα-onp, GalNAcα-pnp, GalNAcβ-pnp, GlcNAcβ-pnp, Galβ-pnp, GlcNAcβ1→3GalNAcα-pnp, L-Fucαal→2Galβ-pnp, GlcNAcα-pnp, Galβ1→6GlcNAcβ-pnp and Galβ1→3GlcNAcβ-pnp.

4. A method according to claim 2 in which the inhibitor of Core 2 GlcNAc-T activity is steroidal glycoside.

5. A method according to claim 2 in which the inhibitor of Core 2 GlcNAc-T activity is selected from the group consisting of Trigoneoside IVa, Clycoside F, Compound 3, Pardarinoside C, Shatavarin I, Shatavarin IV, Deltonin, Balanitin VI, solasodine 3-O-α-L-rhamnopyranosyl-(1→2)-O-[β-D-glucopyranosyl-(1→4)]-β-D-glucopyranoside, Solanidine 3-O-α-L-rhamnopyranosyl-(1→2)-O-[β-D-glucopyranosyl-(1→4)]-β-D-glucopyranoside.

6. A method according to claim 1 wherein the compound capable of reducing the activity of Core 2 GlcNAc-T is an inhibitor of protein kinase Cβ.

7. A method according to claim 6 wherein the compound capable of reducing the activity of Core 2 GlcNAc-T is an inhibitor of protein kinase Cβ.

8. A method according to claim 6 wherein the compound is selected from the group consisting of 3,4-di-indoyl-pyrrol-2,5-dione derivatives.

9. A method according to claim 6 wherein the compound is ruboxystaurin.

10. A method of diagnosing Multiple Sclerosis in a subject comprising comparing the level of Core 2 GlcNAc-T activity associated with samples isolated from the subject with the level of Core 2 GlcNAc-T activity determined in samples isolated from healthy non afflicted individuals, a level of Core 2 GlcNAc-T higher than that in samples isolated from healthy non afflicted individuals being indicative that the subject is afflicted with MS.

11. A method according to claim 10 wherein a level of Core 2 GlcNAc-T activity associated with samples isolated from the subject that is least 2 times higher than that in samples isolated from healthy non afflicted individuals is indicative of MS in the subject.

12. A method of determining the utility of a substance for use in the treatment of Multiple Sclerosis comprising measuring the ability of the substance to inhibit the activity of Core 2 GlcNAc-T.

13. A method according to claim 12 wherein the ability of the substance to inhibit the activity of Core 2 GlcNAc-T is measured by comparing the level of Core 2 GlcNAc-T activity obtained in an assay in which a test substance is incorporated to the level of Core 2 GlcNAc-T activity in the assay with no test substance.

14. A method according to claim 12 wherein the ability of the substance to inhibit the activity of Core 2 GlcNAc-T is measured by a method comprising (a) contacting source of active Core 2 GlcNAc-T enzyme with an acceptor and a sugar donor for a Core 2 GlcNAc-T in the presence of a test substance; (b) measuring the amount of sugar donor transferred to the acceptor, and (c) carrying out steps (a) and (b) in the absence of the test substance to determine whether the substance inhibits the transfer of the sugar donor to the acceptor by the Core 2 GlcNAc-T.
